(19) United States
(12) Patent Application Publication
(10) Pub. No.: US 2022/0176107 A1
(43) Pub. Date: Jun. 9, 2022

(54) PSEUDO-MONOPOLAR NEURAL INTERFACE SYSTEM

(71) Applicant: Galvani Bioelectronics Limited, Brentford (GB)

(72) Inventor: Kenneth Douglas RYS, Brentford (GB)

(21) Appl. No.: 17/593,664

(22) PCT Filed: Mar. 25, 2020
(86) PCT No.: PCT/US20/24646

§ 371 Date: Sep. 22, 2021

Related U.S. Application Data
(60) Provisional application No. 62/823,244, filed on Mar. 25, 2019.

(51) Int. Cl. A61N 1/05; A61N 1/36

(52) U.S. Cl. CPC A61N 1/0556; A61N 1/36053; A61N 1/0558

(57) ABSTRACT

A neural interface system, comprises a lead body including a first electrode; a cuff body having a first section and a second section separated by a gap for providing a path of least electrical resistance, a second electrode positioned within the first section; and a junction between the lead body and the cuff body, wherein the gap extends around an axis of the cuff body and comprises a first area proximal to the junction.

### RELATED APPLICATION

The present application claims the benefit of U.S. Provisional Application No. 62/823,244 filed Mar. 25, 2019, which is hereby incorporated herein in its entirety by reference.

### BACKGROUND

Electrical devices of various shapes and sizes, including one or more electrodes, have been used for neurostimulation of target anatomy for years. One such device, a double-helix cuff used in the CYBERONICS VNS therapy system, is a bipolar device that includes three separate cuffs, one including the anode electrode(s), one containing the cathode electrode(s), and one containing no electrical devices, the later serving only as an anchor tether to the vagus nerve in the neck.

While monopolar lead configurations have been shown to be more efficacious at stimulating nerves than bipolar configurations, it is also generally believed that the stimulation parameters of monopolar cuffs are independent of cuff geometry. Further, monopolar cuffs may exhibit undesirable effects. For example, in Ungar I J., Mortimer J. T., Sweeney J D, “Generation of unidirectionally propagating action potentials using a monopolar electrode cuff,” Annals of Biomedical Engineering 1986; 14(5):437-50.), a monopolar electrode cuff is described that includes a conductor positioned at one end of the cuff and a single cathode located at least 5 mm from an end. This configuration resulted in a unidirectional propagation with minimal current and charge injection. The range of stimulus current values that produced unidirectional propagation increased with increases in longitudinal asymmetry of cathode placement over the range of asymmetries tested.

### SUMMARY

The present disclosure is related to embodiments of a neural interface system for neuromodulation of a target anatomy, such as a neurovascular bundle or an isolated nerve. The neural interface system comprises a lead body including a first electrode; a cuff body having a first section and a second section separated by a gap for providing a path of least electrical resistance, a second electrode positioned within the first section; and a junction between the lead body and the cuff body, wherein the gap extends around an axis of the cuff body and comprises a first area proximal to the junction.

As one of the electrodes is positioned in the lead body rather than in the flexible flaps of the cuff, the cuff's flexibility is enhanced. The lead body is connectable between the cuff body and an implantable pulse generator. The gap, which provides a path of least electrical resistance, positioned in the flaps and between the first and second electrodes may cause a current supplied to be steered through the gap.

The gap may be preformed. In some embodiments, the first section and the second section may be separable by a gap.

The gap may extend at least 180-degrees around an axis of the cuff body, optionally the gap may extend at least 270-degrees around an axis of the cuff body. Thus, the gap may be shaped to cover at least 270-degrees around the target anatomy so that current can be directed to a large fraction of the circumferential portions of a nerve which is the target or circumferentially distributed nerves of the target.

The second electrode may comprise a central electrode portion substantially opposite the first area of the gap proximal to the junction. This arrangement may promote the current to be steered to cover most of the circumference of the target. The second electrode may be positioned in other positions depending on the preferred current path.

The cuff body may be cylindrical. The targets comprise a generally cylindrical shape and thus a substantially cylindrical cuff body may easily conform around the target.

The first and second sections may be formed of an electrically insulative material, optionally wherein the electrically insulative material comprises at least one of: silicone, polyurethane, and copolymers of both.

The first electrode may be configured to be a return electrode and the second electrode may be configured to be a stimulating electrode when the system is configured to stimulate, and the first electrode may be configured to be a stimulating electrode and the second electrode may be configured to be a return electrode when the system is configured to block. Thus, the neural interface system may be used for both (if required) stimulation (i.e., start propagation of an action potential) and blocking (i.e., stop propagation of action potential).

A surface area of the first electrode may be larger than a surface area of the second electrode, optionally the ratio between the first electrode area to the second electrode area may range between 3:1 to 10:1, further optionally the ratio between the first electrode area to the second electrode area may be 5:1.

The second section may be an anchoring device for at least partially maintaining contact between the second section and a target nerve or neurovascular bundle or neurovascular vessel to be stimulated.

The first electrode may be configured to not extend beyond a point where the lead body joins the junction. Thus, a sufficient distance between the first electrode and the second electrode may be provided to mimic the properties of a monopolar configuration, whilst the first and second electrodes are close enough to each other to contain the current flow to intended target area.

The first electrode may be a flexible coil electrode.

The first electrode may be a ring electrode.

The second electrode may be a single electrode and occupy at least 180 degrees around the axis of the cuff body thereby extending at least half of a circumference of the circular cuff body, optionally the second electrode may occupy at least 225 degrees around the axis of the cuff body.

The second electrode may be a segmented electrode and occupies approximately 180 degrees of a circumference of the circular cuff body thereby extending at least half of a circumference of the circular cuff body, optionally the second electrode may occupy at least 225 degrees around the axis of the cuff body, further optionally the second electrode may occupy at least 270 degrees around the axis of the cuff body.

The second electrode may be substantially normal to the gap.

The second electrode may form a partial cylinder.

The cuff body may have a length and the lead body may be substantially perpendicular to the length.

The cuff body may have a length and the lead body is substantially parallel to the length.

The gap may start at a proximal end of the cuff body and extends along a length of the cuff body until past a distal end of the cathode, whereupon the gap turns and is configured to form the second section.

The gap may start at a proximal end of the cuff body proximal the junction and extend along a length of the cuff body until turning at least 270-degrees to from the second section.

The lead body may be substantially parallel to a length of the cuff body, and the gap may start at a distal end of the cuff body proximal the lead body and extend along the length of the cuff body until turning at least 270-degrees to form the second section.

### BRIEF DESCRIPTION OF THE SEVERAL VIEWS OF THE DRAWING

FIG. 1 is a perspective view of an embodiment of a pseudo-monopolar cuff;

FIG. 2 is a side view of an embodiment of a coil anode for use with the cuff of FIG. 1;

FIG. 3A is a first side view of the embodiment of FIG. 1;

FIG. 3B is a second side view opposite the first side view of FIG. 3A;

FIG. 4A is a cross-sectional end view of an embodiment the pseudo-monopolar cuff;

FIG. 4B is a cross-sectional view of the embodiment of FIG. 4A simulating current being steered through the gap closes to the anode;

FIG. 4C is a cross-sectional view of the embodiment of FIG. 4B simulating how stimulated regions of the target approach a 360-degree area around the target;

FIG. 5 is a perspective view of an embodiment where a connecting lead is parallel to the cuff;

FIG. 6A is a side view of another embodiment of a connecting lead parallel to the cuff;

FIG. 6B is a left side perspective view of the embodiment of FIG. 6A; and

FIG. 6C is a right side perspective view of the embodiment of FIG. 6A.

### DETAILED DESCRIPTION

The disclosure is directed to a cuff device for neuromodulation that may act like a monopolar configuration. In a monopolar configuration, the physical separation between the anode and the cathode may be much greater than the dimensions of the target where the cuff is deployed.

The present neural stimulation cuff device comprises a first electrode (which may be a stimulating electrode, for example a cathode) positioned in the cuff and a second electrode (which may be a return electrode, for example an anode) proximal to the cuff (but not in the cuff) and within the dimensions of the target, so it is technically bipolar. Hence, the cuff is referred to herein as a pseudo-monopolar because it is bipolar but has operational characteristics of a monopolar cuff.

These operational characteristics may be achieved by positioning the cathode in the cuff, with the anode formed in the lead body connected to the cuff and terminating just proximal to the junction with the cuff. The anode (in the form of a flexible coil similar to an implantable cardioverter defibrillator lead) may have a surface area that is large relative to the cathode, for example 3× or larger, 5× or larger, or 10× or larger, which may offset increases in geometric impedance, improve capacitance, decrease anodic current density, and enable the neural stimulation cuff to be tuned to meet particular impedance/capacitance profiles. By providing the first electrode in the lead body, the cuff body may be used solely for placement of the second electrode. This leads to decrease in design restrictions for the second electrode, as well as potentially increased flexibility in the cuff body (as the absence of the first electrode in the cuff body may lead to less electrode portions being present in the cuff body).

In some embodiments, the anode may be positioned in the cuff and the cathode may be positioned in the lead body. In other words, although many of the embodiments are described in the present disclosure with the cathode being positioned in the cuff and the anode being positioned in the lead body, the first electrode in the cuff and the second electrode in the lead body may be configured to be a stimulating electrode or a return electrode depending on the requirement. For example, the first electrode may be configured to be a return electrode and the second electrode may be configured to be a stimulating electrode when the neural stimulation cuff device is configured to stimulate (for example by inducing depolarization in the target), and wherein the first electrode is configured to be a stimulating electrode and the second electrode is configured to be a return electrode when the device is configured to block (for example by inducing hyperpolarization in the target). The cathode may be positioned along a portion of the interior diameter covering perhaps 180 degrees around the axis of the cuff body. A central portion of the cathode may be positioned substantially perpendicular to the gap closest to the anode, as further described below.

The physical cuff may include two or more flaps (or arms) extending in opposing directions such that a gap (also referred to as an opening or space which may at least partially be filled with body fluid or tissue) may be formed between the flaps. For example, said flaps may be created by cutting a single flap. The flaps may be shaped so that the gap between the flaps may cover or be adjacent to at least a 270-degree to a nearly 360-degree area around the target. The flaps may be curved and the two flaps or arms extending in opposing directions may form a substantially cylindrical shape of the cuff body. Only one flap may have an active electrode, with the other flap performing mainly a fixation or support function. In some embodiments, both (or all, if more than two flaps are provided) flaps may have an active electrode. The flap with an active electrode may also perform a fixation or support function. Additional fixation or support flaps may also be provided. Removing the anode (or the first electrode) from the active flap of the cuff may allow the interface to be significantly softer (i.e., because inflexible metallic electrodes are removed) and reduce the number of connections that need to be formed between leads and the cathode electrodes (or the second electrodes) in the cuff.

When current is supplied to the cathode (or the second electrode), which may be a singular electrode or a segmented electrode, current may seek to flow from the anode (or the first electrode) to the nearby cathode, but because the flaps of the cuff are nonconductive, the current may first flow through the gaps (i.e., a path of least electrical resistance). Thus, the path of current flow can be adjusted depending on the formation of the gap. Where the gap is formed almost 360 degrees about an axis of the target, this may cause current to flow almost 360 degrees about an axis of the target, such that the current flows almost fully around the outside or inside of the target around or within which the cuff is positioned in order to get to the cathode. The shape or position of the gap in combination with the shape or position of the second electrode in the flap may be configured in order to promote different paths of least resistance for the current to flow through. Thus, the gap positioned between the first and second electrodes may be configured to adjust the path of least resistance for the current depending on the desired activation of the target (or even depending on the desired activation portions of the target). The cuff being nonconductive means that the cuff has a significantly higher impedance compared to the cuff surroundings such as surrounding body tissue. For example, the cuff may be formed of silicone, polyurethane, or copolymers of both, and material resistivity of the cuff may be around 1 Ωm-10 Ωm. Such current steering, where “current steering” is used here to mean that current is driven in a certain direction by providing a preferred path with a lower resistance, using a gap positioned between the first and second electrode promotes the current to pass across tissue containing the nerves of interest, potentially irrespective of the magnitude of fibrotic encapsulation that may occur in areas covered by or adjacent to the flaps. Here, the phrase “current steering” means providing a predefined path of least resistance for the current, which may also be referred to as a path by which the current preferentially travels. In other words, current is driven in a certain direction by providing a preferred path with a lower resistance, where this preferred path is provided by a gap in an electrically insulative material. A further benefit may be that the disclosed cuff device requires lower power levels than bipolar devices, which may improve safety, increase patient tolerance, and reduce chronic impedance increases associated with fibrosis.

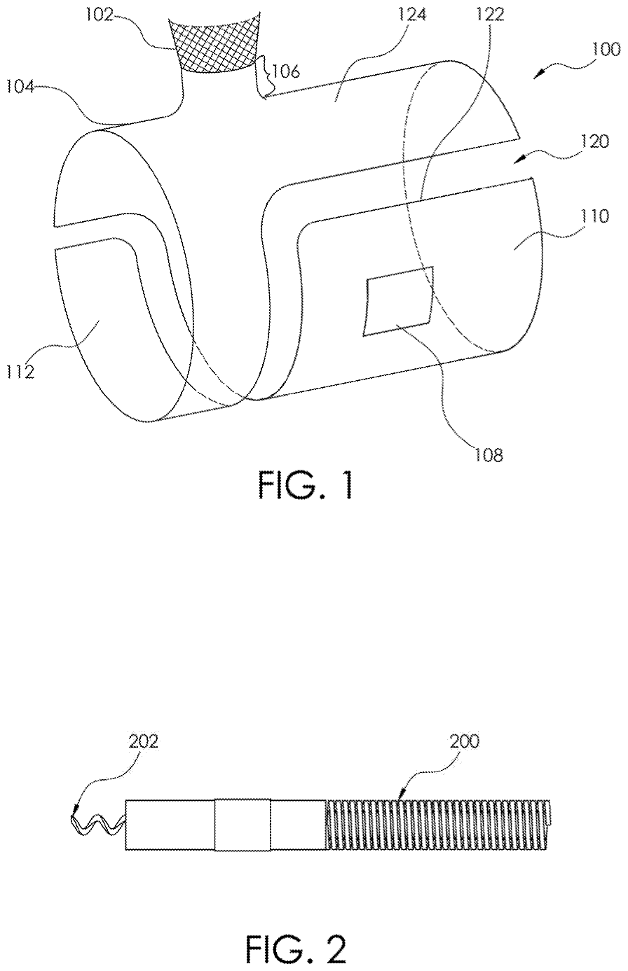

A neural interface device 100 in accordance with an embodiment is illustrated in FIG. 1 and FIG. 2. The device 100 may generally consist of a connecting lead 102 attached to a cuff 104. The anode 102 (negative return electrode) and wiring for connection to a cathode 108 (positive stimulating electrode) may be incorporated into the body of the connecting lead. The anode 102 may be a flexible coil, such as coil 200 illustrated in FIG. 2, which may be similar to an implantable cardioverter defibrillator lead of a type that may be readily available. Rigid ring electrodes may also be used for the anode 102. The coil 200 may terminate proximal to a junction 106 between the connecting lead 102 and the cuff 104. The wiring 202 may protrude from the connecting lead 102 at the junction 106 and be routed through the cuff 104 to connect to the cathode 108. The anode may therefore be removed from the neural interface portion of the device entirely. Removing the anode 200 from the cuff 104 may allow the cuff 104 to be significantly softer, as inflexible metallic electrodes may be removed in the process. This may have the added benefit of removing further wiring for the anode from the cuff 104, as well as the need to provide delicate interconnections to that wiring. This may reduce the complexity of fabricating the device and improve its reliability.

The surface areas of the anode and cathode may be asymmetric. That means the anode 200 may have a surface area that is very large relative to the cathode 108, perhaps as much as 10 times larger. For example, the surface area ratio between the first electrode (for example the anode) and the second electrode (for example the cathode) ranges between 3:1 to 10:1 (the surface area of the first electrode being larger than that of the second electrode, where the ratio between the first electrode area to the second electrode area may be 5:1. As the size of the anode 200 may not be constrained by the geometry of the cuff 104, the anode 200 may be arbitrarily large, but may still be positioned close enough to the cathode 108 to offset increases in geometric impedance. In other words, the anode (first electrode) is positioned optimally, taking into account the trade-off between being further away from the cathode (second electrode) to achieve monopolar characteristics versus being closer to the anode for reduced impedance. Considering the anode as being positioned between the implanted pulse generator and the cathode, the anode may be positioned at a position where the distance between the anode and the cathode is less than the distance between the anode and the IPG. Preferably, the anode is significantly closer to the cathode than to the IPG, for example around the end portion of the lead body connecting to the cuff body. In such a configuration, the current flow is better controlled (reaching the target issue more directly) and the impedance due to the distance between the cathode and the anode is minimized such that the current required is not high enough to significantly damage the surrounding or target tissue. The size of the anode may also serve to improve capacitance and decrease anodic current density. In other words, the present disclosure may make it easier to tune the device to meet a particular impedance/capacitance profile, and especially those requiring low system impedance.

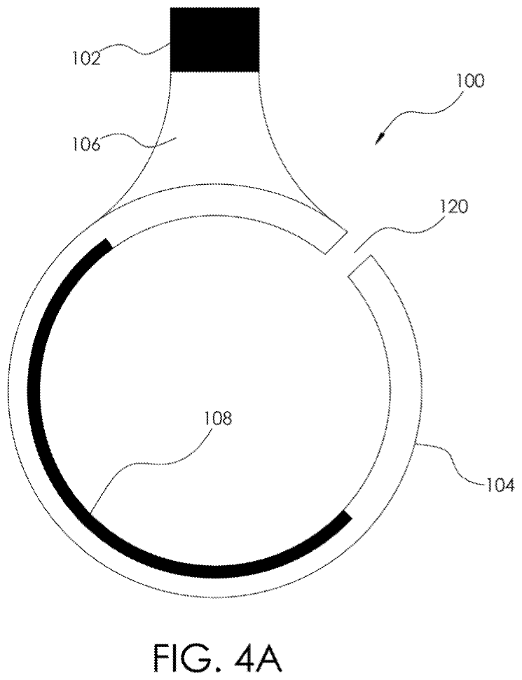

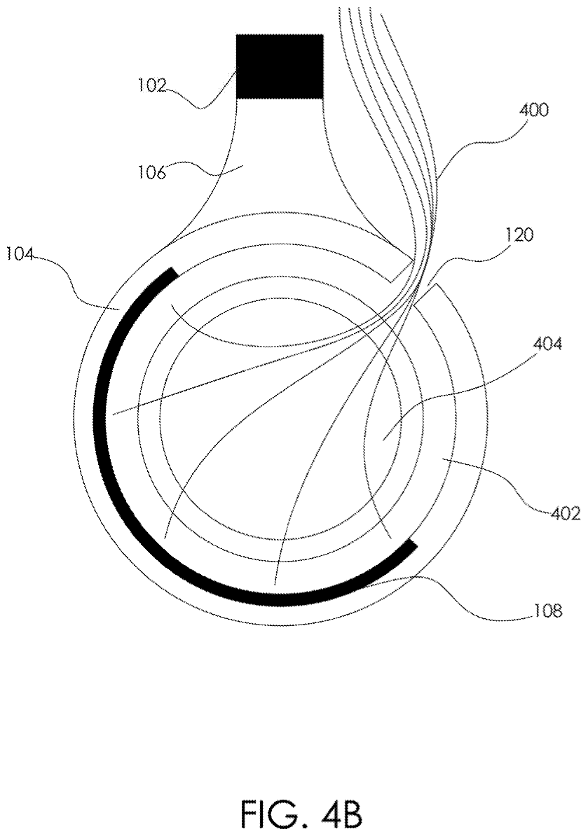

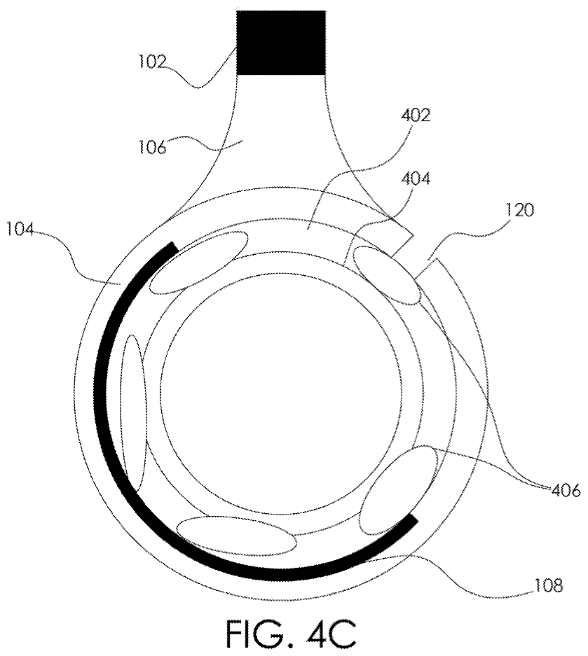

As illustrated in FIG. 1, the cuff 104 may have two flaps or rings, an active flap 110 that may contain the cathode 108 and one or more additional inactive or passive flap(s) 112 that may be configured to serve as anchors or fixation devices. The additional passive flap(s) 112 may not include any functional electrical devices and may also just support the fixation of the device. A gap 120 may be formed to start at an end 122 of the active flap 110 and run between the active flap 110 and a portion 124 of the cuff 104 proximal the junction 106. The gap 120 may be as close to the junction 106 as possible, as further illustrated in FIG. 4A, which may be referred to herein as being “clocked” as the gap may be close to a 12 o'clock position when turned so the gap is up when viewed from a proximal end of the cuff 104. For example, a junction connecting the lead body and the cuff body may be considered as the zero position (or 12 o'clock position). Thus, the cathode being positioned opposite the lead or substantially normal may be understood as the cathode being positioned at 6 o'clock position or 180 degrees from the zero position. Other initial proximal clocking positions may be possible and desirable for the gap 120. The gap 120 may then proceed from a proximal end along a length of the cuff 104, past the junction 106 to a position just beyond the distal end of the junction 106, where it may turn and rotate between at least 270-degrees to just less than 360-degrees around the cuff 104, where it may turn again and terminate at the distal end of the cuff 104.

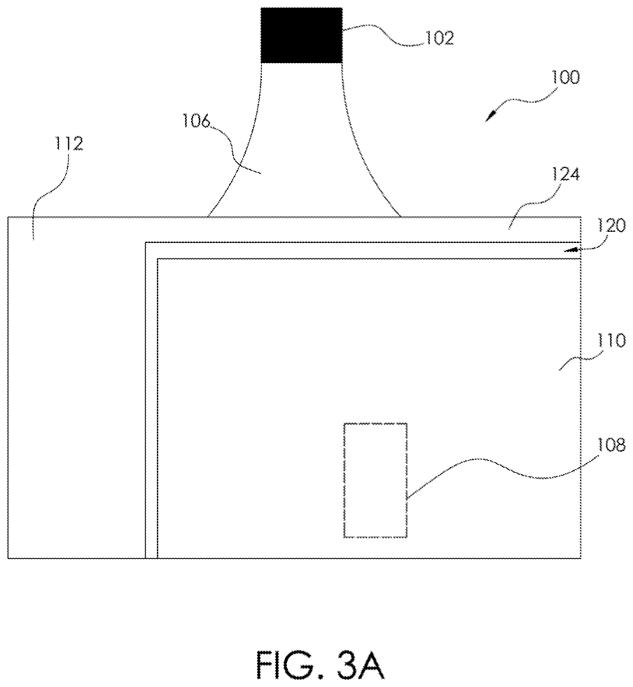

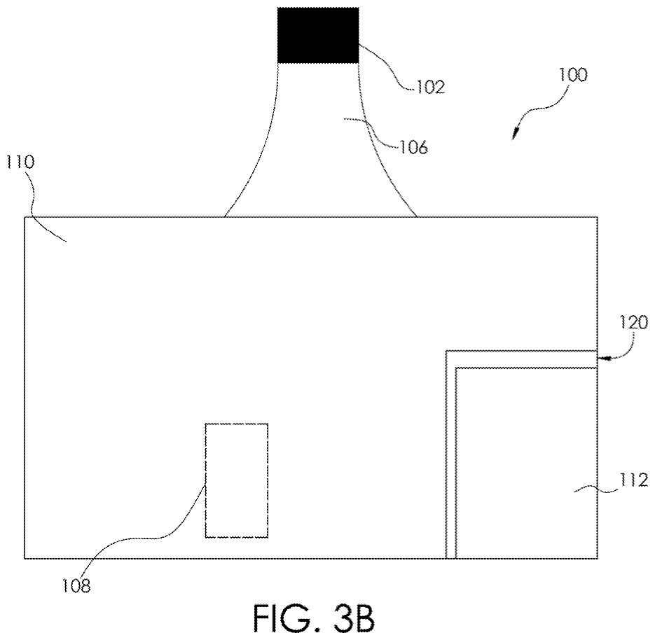

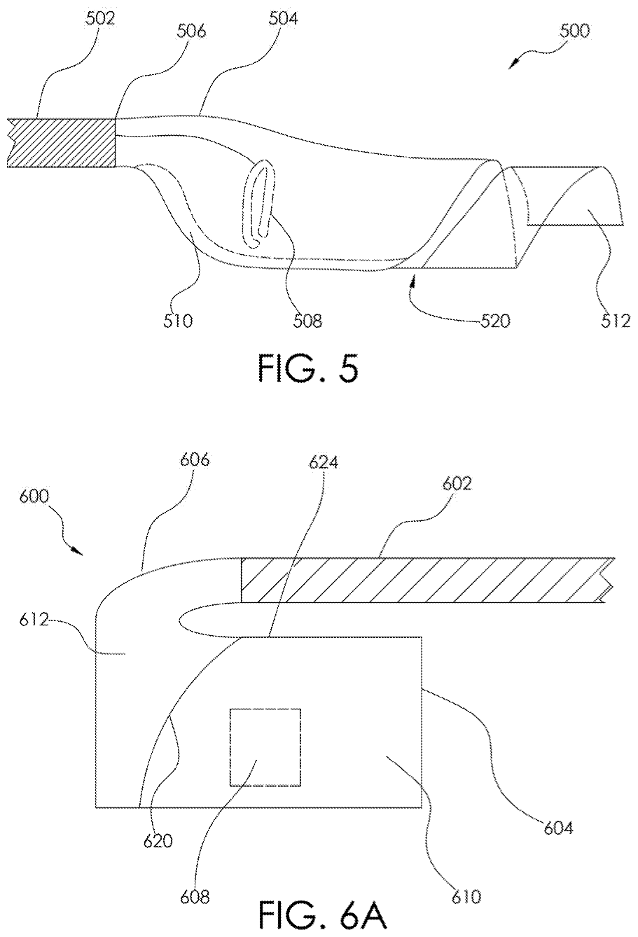

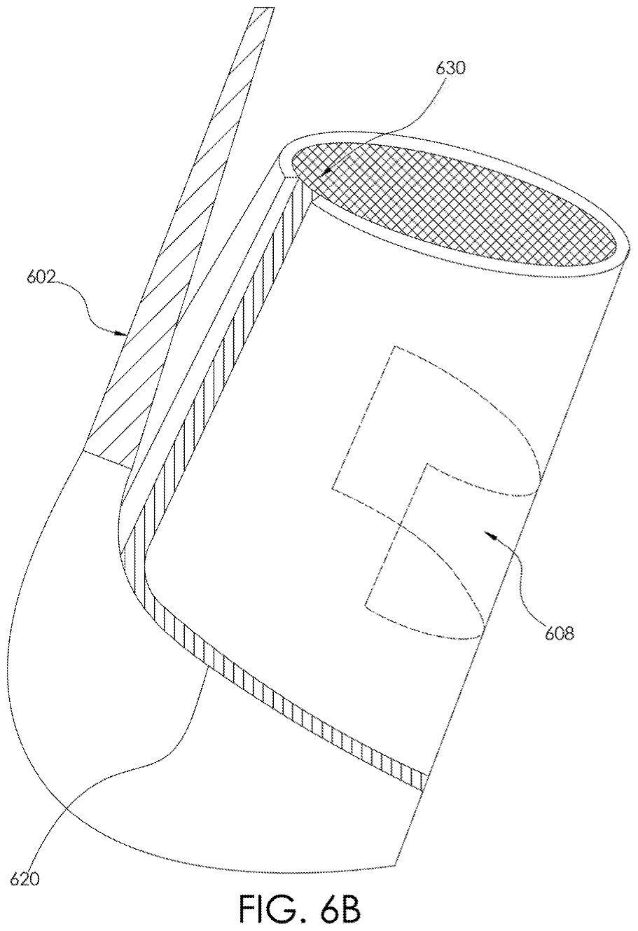

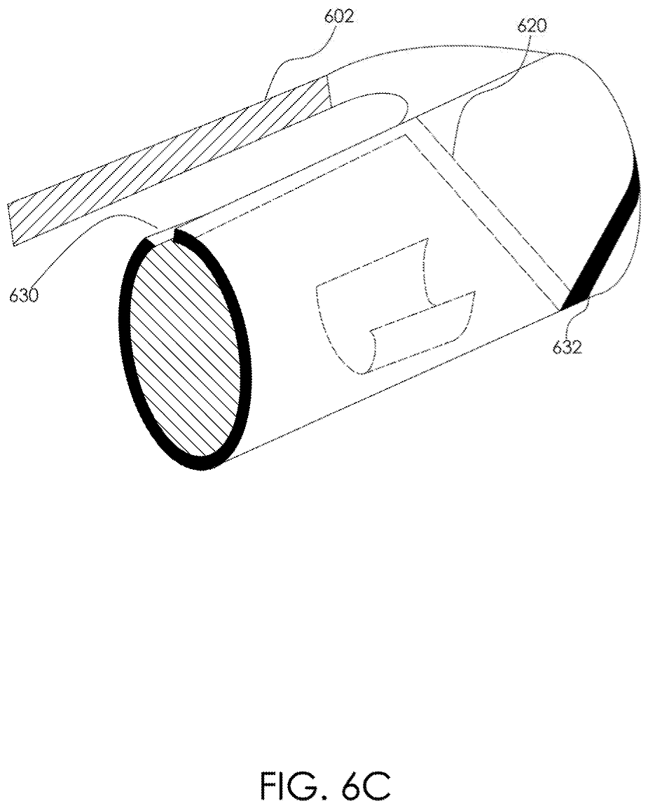

The cathode 108 may occupy an area constituting a fractional arc of the circumference or inner/outer diameter of the cuff 104, such as up to 180 degrees, and that area may be oriented to be “substantially normal,” as further described below, to the gap 120 at the 12 o'clock position. The positions of the cathodes 108, 508 and 608 in FIG. 1, FIG. 3A, FIG. 3B, and FIG. 5, and FIG. 6A, respectively, are merely illustrative and do not necessary illustrate the cathode positioned as described immediately above. In a cuff 104 with an 8 mm internal diameter, the cathode may have a width of 1.6 mm, which may yield a cathode surface area of 20 mm² and provide a Shannon safety k-factor of approximately 2.0 at 20 mA, although many other dimensions may work if balanced appropriately.

The configuration of the cathode 108 and anode 200 relative to one another may approximate a “monopolar” configuration where the anode and cathode are separated by a distance that is much greater than the dimensions of the target to be simulated. The neural interface device may be referred to herein as “pseudo-monopolar” for this reason. As monopolar lead configurations have been shown to be up to 10 times more efficacious at stimulating nerves in vivo than bipolar configurations, and require lower power levels, this present disclosure may be appropriate for splenic neurovascular bundle stimulation.

The gap 120 may also create a path of least resistance between the cathode 108 and the anode 200, which may cause current flowing between the cathode 108 and the anode 200 to be steered through and along the gap 120. As the gap 120 covers between at least 270-degrees to almost 360-degrees of the inner diameter of the cuff 104, current may pass along a large fraction of the circumference (inner/outer diameter) of the neurovascular bundle being stimulated in a neurovascular embodiment. Furthermore, as the current may be steered through the gap 120 over neurovascular bundle tissue that may not be contacted by the cuff 104, as opposed to a bipolar cuff where current primarily flows along contacted portions of the tissue, the current may always pass across tissue that contains nerves of interest, irrespective of the magnitude of fibrotic encapsulation that may occur in the contacted tissue areas. FIG. 3A is a side view of an extravascular device 100 further illustrating the position of the gap 120 between the active flap 110 and the inactive flap 112, as well as the relative positioning of the anode 102 and cathode 108. FIG. 3B is an opposite side view of the same.

For an isolated nerve embodiment, the side of the device may be further reduced for positioning around the nerve, such as for use in direct vagus nerve stimulation. In an isolated nerve application, the gap may cover between at least 270 degrees and 360 degrees of the inner/outer diameter of the cuff so current may pass through a large fraction of the nerve cross-section.

FIG. 4A illustrates a cross-sectional end view of an extravascular device 100 further illustrating the relative positioning of the anode 102 and cathode 108 such that the cathode 108 is more clearly shown to be configured in a position within the cuff 104 that may be considered “substantially normal” (given that the cuff is an arc) or substantially opposite to the gap 120 at the 12 o'clock position. As noted, the cuff 104 is substantially circular and has an internal diameter that is parallel to the neurovascular bundle around which it is to be positioned. The cathode 108, which may be positioned along a portion of the interior or exterior diameter of the cuff 104 may therefore form an arc that may occupy approximately 180-degrees of the diameter of the cuff 104. Although the cathode 108 is referenced herein as being “substantially perpendicular” or “substantially normal” to the gap 120, because the cathode 108 is not planar it may not be perpendicular or normal, per se, to the gap 120. Nevertheless, a center portion of the arc of the cathode may be positioned substantially opposite the gap 120, which would therefore position the cathode perpendicular or normal to the gap 120, if the cathode were planar about that center portion.

As shown in FIG. 4B, which is a simulation of how current may be steered through the gap 120 closest to the anode 102, current 400 may be simulated flowing from the anode 102, through the gap 120 in the cuff 104, and into the nerve 402 surrounding a target which in this example is a blood vessel 404. As further simulated in FIG. 4C, the current 400 flow may stimulate a number of different regions 406 of the nerves 402, approaching a 360-degree area around the blood vessel 404.

Additional embodiments of the device 100 are shown in FIG. 5 and FIG. 6A, FIG. 6B and FIG. 6C. In FIG. 5, the device 500 may include an anode 502 located in a lead that runs parallel to the cuff 504. The anode 502 may terminate proximal the junction 506 between the lead and the cuff 504. The cuff 504 may include two flaps, an active flap 510 and a passive flap 512. The active flap 510 may include the cathode 508 that may cover about 180-degrees of the interior or exterior diameter of the active flap 510 and which may be positioned substantially normal the gap 520 formed between the active flap 510 and the passive flap 512. The gap 520 may start proximal the junction 506 proximal an underside of the cuff 504, run or travel past the cathode 508 to a point where the active flap 510 and passive flap 512 diverge, and then wrap around the device 500 by at least 270-degrees or more. In essence, the active flap 510 and passive flap 512 may travel together along opposite sides of the gap 520 until the passive flap 512 separate and the passive flap curves around on its own. The device 500 may be configured as a fixation device around the stimulation target.

In other words, the cuff 504, when viewed from the distal end, may project proximally from the junction 506 and have a single gap 520 that may begin at close to a 12 o'clock position proximally (just below the junction 506), drop down to a 6 o'clock position and then extend straight along the bottom of the cuff 504 until passing the distal edge of the cathode 508, where shortly thereafter it may spiral approximately 270-degrees through a 9 o'clock position so as to terminate at the distal edge around an almost 3 o'clock position. The passive flap 512 created by the spiral gap 520 may serve, to provide additional fixation.

FIG. 6A is a side view of another embodiment of a device 600 having a connecting lead parallel to the cuff. In this embodiment, the anode 602 in the lead runs alongside a portion of the cuff 604 until proximal the junction 606. The cuff 604 formed after the junction 606 may include two flaps, an active flap 610 (including the cathode 608) and a passive flap 612 separated by a gap 620 that runs along a substantial length of an upper portion 624 of the cuff 604 until the gap 620 reaches the junction 606, at which point air gap 620 curves around and creates the separate flaps 610 and 612. FIG. 6B is a left side perspective view of the device 600 better illustrating the cathode 608 and the gap 620 as it starts at starting point 630, traverses straight along the line of the anode 602 and then spirals along the left side of the device 600. FIG. 6C is a right side perspective view further illustrating the gap 620 from its starting point 630 to its ending point 632. As in other embodiments, the cathode 608 may be positioned substantially normal to the opening 620.

The single gap 620 of the cuff may start at a distal end 630 of the cuff 604 that begins at a 12 o'clock position, when viewed from the distal end beneath an upper portion 624 of the cuff 604, and extend along the cuff 614 until passing the proximal edge of the cathode 608, where the gap 620 spirals toward a 9 o'clock position and continues to an almost 3 o'clock position, thereby creating the passive flap 612, which may be used for additional fixation. In the embodiment, the proximal cuff insulation may be beveled at approximately 45-degrees to minimize geometric current shunting. The cathode 608 may cover 180-degrees along the inner/outer diameter of the cuff 604 and be centered at about 6 o'clock. The edges of the cathode 608 may be filleted, if possible, to eliminate excessive charge concentration. For a 5 mm interior diameter cuff 604, the cathode area may be approximately 20 mm² when the cathode width is 2.5 mm. The total interface length may be about 12.5 mm. Other dimensions may naturally be utilized.

In an embodiment, a neurovascular device comprising a lead body including an anode, a circular cuff body having a first section and a second section separated by a gap, a cathode positioned within the first section, and a junction between the lead body and the cuff body, wherein the gap extends at least 270-degrees around the axis of the circular cuff body and comprises a first area proximal to the junction, the cathode having a second area substantially opposite the first area of the gap proximal to the junction. In the embodiment, wherein the second section is a fixation device for an extravascular application, and wherein the fixation device is configured to fixate the second section to a nerve, or neurovascular bundle or vessel to be stimulated. In the embodiment, wherein in the anode does not extend beyond a point where the lead body joins the junction. In the embodiment, wherein the anode is a flexible coil electrode. In the embodiment, wherein the anode is a ring electrode.

In the embodiment, wherein the cathode is a single electrode and the second area occupies approximately 180-degrees of a circumference of the circular cuff body. In the embodiment, wherein the cathode is substantially normal to the gap. In the embodiment, wherein the cathode is a segmented electrode and the second area occupies approximately 180 degrees of a circumference of the circular cuff body. In the embodiment, wherein the cathode is substantially normal to the gap.

In the embodiment, wherein the cathode forms an arc. wherein the circular cuff body has a length and the lead body is substantially perpendicular to the length. In the embodiment, wherein the circular cuff body has a length and the lead body is substantially parallel to the length. In the embodiment, wherein the gap starts at a proximal end of the circular cuff body and extends along a length of the circular cuff body until past a distal end of the cathode, whereupon the gap turns and is configured to form the second section.

In the embodiment, wherein the gap starts at a proximal end of the circular cuff body proximal the junction and extends along a length of the circular cuff body until turning at least 270-degrees to from the second section.

In the embodiment, wherein the lead body is substantially parallel to a length of the circular cuff body, and wherein the gap starts at a distal end of the circular cuff body proximal the lead body and extends along the length of the circular cuff body until turning at least 270-degrees to form the second section.

The embodiments of the present disclosure, while illustrated and described in terms of various embodiments, is not limited to the particular description contained in this specification. Additional alternative or equivalent components and elements may be readily used to practice the present disclosure.

1. A neural interface system, comprising:
a lead body including a first electrode;
a cuff body having a first section and a second section separated by a gap for providing a path of least electrical resistance, a second electrode positioned within the first section; and
a junction between the lead body and the cuff body,
wherein the gap extends around an axis of the cuff body and comprises a first area proximal to the junction.

2. The neural interface system of claim 1, wherein the gap extends at least 180-degrees around an axis of the cuff body, optionally wherein the gap extends at least 270-degrees around an axis of the cuff body.

3. The neural interface system of claim 1, wherein the second electrode comprises a central electrode portion substantially opposite the first area of the gap proximal to the junction.

4. The neural interface system of claim 1, wherein the cuff body is cylindrical.

5. The neural interface system of claim 1, wherein the first and second sections are formed of an electrically insulative material, optionally wherein the electrically insulative material comprises at least one of: silicone, polyurethane, and copolymers of both.

6. The neural interface system of claim 1, wherein the first electrode is configured to be a return electrode and the second electrode is configured to be a stimulating electrode when the system is configured to stimulate, and wherein the first electrode is configured to be a stimulating electrode and the second electrode is configured to be a return electrode when the system is configured to block.

7. The neural interface system of claim 1, wherein a surface area of the first electrode is larger than a surface area of the second electrode, optionally wherein the ratio between the first electrode area to the second electrode area ranges between 3:1 to 10:1, further optionally wherein the ratio between the first electrode area to the second electrode area is 5:1.

8. The neural interface system of claim 1, wherein the second section is an anchoring device for at least partially maintaining contact between the second section and a target nerve or neurovascular bundle or neurovascular vessel to be stimulated.

9. The neural interface system of claim 1, wherein in the first electrode does not extend beyond a point where the lead body joins the junction.

10. The neural interface system of claim 1, wherein the first electrode is a flexible coil electrode.

11. The neural interface system of claim 1, wherein the first electrode is a ring electrode.

12. The neural interface system of claim 1, wherein the second electrode is a single electrode and occupies at least 180 degrees around the axis of the cuff body thereby extending at least half of a circumference of the circular cuff body, optionally wherein the second electrode occupies at least 225 degrees around the axis of the cuff body.

13. The neural interface system of claim 1, wherein the second electrode is a segmented electrode and occupies approximately 180 degrees of a circumference of the circular cuff body thereby extending at least half of a circumference of the circular cuff body, optionally wherein the second electrode occupies at least 225 degrees around the axis of the cuff body, further optionally wherein the second electrode occupies at least 270 degrees around the axis of the cuff body.

14. The neural interface system of claim 1, wherein the second electrode is substantially normal to the gap.

15. The neural interface system of claim 1, wherein the second electrode forms a partial cylinder.

16. The neural interface system of claim 1, wherein the cuff body has a length and the lead body is substantially perpendicular to the length.

17. The neural interface system of claim 1, wherein the cuff body has a length and the lead body is substantially parallel to the length.

18. The neural interface system of claim 1, wherein the gap starts at a proximal end of the cuff body and extends along a length of the cuff body until past a distal end of the cathode, whereupon the gap turns and is configured to form the second section.

19. The neural interface system of claim 1, wherein the gap starts at a proximal end of the cuff body proximal the junction and extends along a length of the cuff body until turning at least 270-degrees to from the second section.

20. The neural interface system of claim 1, wherein the lead body is substantially parallel to a length of the cuff body, and wherein the gap starts at a distal end of the cuff body proximal the lead body and extends along the length of the cuff body until turning at least 270-degrees to form the second section.
